(19) United States
(12) Patent Application Publication
(10) Pub. No.: US 2025/0248922 A1
(43) Pub. Date: Aug. 7, 2025

(54) METHOD OF PREPARING A MASTOCARPUS STELLATUS EXTRACT

(71) Applicant: GIVAUDAN SA, VERNIER (CH)

(72) Inventors: Anne HUMEAU, Ile Grande (FR); Marie MEUNIER, Pomacle (FR); Amandine SCANDOLERA, Pomacle (FR); Audrey DE BIZEMONT, Ile Grande (FR); Romain REYNAUD, Toulouse (FR)

(21) Appl. No.: 18/854,121

(22) PCT Filed: May 4, 2023
(86) PCT No.: PCT/EP2023/061743

§ 371 Date: Oct. 4, 2024

(30) Foreign Application Priority Data
May 5, 2022 (GB) 2206529.6
Jul. 19, 2022 (GB) 2210574.6

(51) Int. Cl. A61K 8/9717; A61K 36/04; A61Q 19/06; A61Q 19/08; B01D 11/02

(52) U.S. Cl. CPC A61K 8/9717; A61K 36/04; A61Q 19/06; A61Q 19/08; B01D 11/0288; A61K 2236/15; A61K 2236/17; A61K 2236/331; A61K 2236/53; A61K 2800/782; A61K 2800/805

(57) ABSTRACT

A method of preparing a Mastocarpus stellatus extract is disclosed, as well as cosmetic uses of the extract.

The present invention relates to a method of preparing a Mastocarpus stellatus extract, to the Mastocarpus stellatus extract thus obtained, to a cosmetic composition comprising the Mastocarpus stellatus extract, and to its use in cosmetics.

Mastocarpus stellatus, commonly known as carrageenan moss or false Irish moss, is a species in the Rhodophyceae division, a red algae seaweed division. Mastocarpus stellatus occurs commonly on rocks in the mid and lower-intertidal. It is generally found on most coasts of Ireland and Britain. Other recorded locations include: France (English Sea Channel, North-East Atlantic), Iceland, Faeroes, North Russia to Rio de Oro, Canada (Newfoundland) to U.S. (North Carolina).

Mastocarpus stellatus grows from a discoid holdfast stipe, and the fronds are channeled unlike those of Chondrus crispus, which are flat. It grows to a height of 10-20 cm and branches dichotomously. The frond is cartilaginous and reddish-brown in color, with a greenish or purplish tinge. The mature algae show reproductive structures which develop on erect filaments up to 1 mm in diameter. In color it is reddish brown, purple or bleached.

The earliest record of collecting Irish seaweed is evidenced by 12th century poems by monks, according to Michael Guiry. In a 2001 market analysis of Irish seaweed aquaculture, the estimated combined annual national seaweed harvest of M. stellatus and C. crispus was less than 100 tons. M. stellatus is harvested during the gametophyte life phase because later phases, with more sulphated carrageenan, are harder to remove from its rock. The food and pharmaceutical industries are interested in the seaweed for their antioxidant, anticoagulant, and thickening or gelling properties. In addition to its health properties and applications, the gelling properties of M. stellatus can create a biodegradable film that may be a sustainable and edible alternative to plastics for food preservation and functional food development.

EP 1 743 628 A1 relates to a cosmetic composition comprising a red algae extract comprising a combination of 25-50 wt % of floridoside and 10-25 wt % of isethionic acid with a mass ratio of 1:5, and a carrier. The composition may be used to hydrate the skin and/or prevent skin ageing.

FR 2 946 878 B1 relates to the cosmetic use of floridoside or a red algae extract as a melanogenesis inhibitor.

Surprisingly, it has now been found that by using a special extraction method, it is possible to obtain a Mastocarpus stellatus extract that has a lypolytic activity and is able to increase skin elasticity, thus allowing for reducing a double chin.

Therefore, in a first aspect, the present invention provides a method of preparing a Mastocarpus stellatus extract. The method comprises the following steps:
(i) Aqueous extraction of Mastocarpus stellatus to obtain a first liquid phase and a first solid phase;
(ii) Acidic hydrolysis of the first solid phase at elevated temperature;
(iii) Phase separation to obtain a second liquid phase and a second solid phase; and
(iv) Combining the first liquid phase and the second liquid phase to obtain the Mastocarpus stellatus extract.

In a second aspect, the present invention relates to a Mastocarpus stellatus extract obtained by or obtainable by the method of the invention.

In a third aspect, the present invention relates to a cosmetic composition comprising the Mastocarpus stellatus extract of the invention and a cosmetically acceptable excipient.

In a fourth aspect, the present invention relates to a method of reducing a double chin, comprising the step of topically applying a Mastocarpus stellatus extract, and in particular the Mastocarpus stellatus extract the invention or the cosmetic composition of the invention, to the double chin.

In a fifth aspect, the present invention relates to the use of the Mastocarpus stellatus extract of the invention or of the cosmetic composition of the invention for reducing a double chin, for enhancing lipolysis, for increasing skin elasticity, for stimulating collagen synthesis, for hydrating skin, for reducing the effects of skin aging and/or for preventing the effects of skin aging.

The invention and its preferred features will be described in more detail below.

For the avoidance of doubt, preferences, options, particular features and the like indicated for a given aspect, feature or parameter of the invention should, unless the context indicates otherwise, be regarded as having been disclosed in combination with any and all other preferences, options, particular features and the like as indicated for the same or other aspects, features and parameters of the invention.

The method of the invention comprises an aqueous extraction of Mastocarpus stellatus to obtain a first liquid phase and a first solid phase.

The aqueous extraction may be performed on entire Mastocarpus stellatus plants. Alternatively, it is also possible to use only certain parts of the plants, for example the stipe and/or fronds.

Prior to the aqueous extraction, the Mastocarpus stellatus may be dried and/or processed into smaller pieces, for example by cutting or grinding or crushing or mincing or any other suitable method known to the skilled person.

Therefore, in an embodiment, the Mastocarpus stellatus is dried prior to the aqueous extraction. The algae may be dried by any suitable method, e.g. in an oven at low temperature (e.g. about 40° C.) or in the sun. Using dried algae facilitates storage and logistics. Alternatively, it is also possible to use fresh algae or frozen algae, for example.

In an embodiment, the Mastocarpus stellatus is processed into smaller pieces, in particular cut and/or ground, prior to the aqueous extraction. This improves the efficacy of the extraction process. Preferably, the Mastocarpus stellatus is processed into pieces of about 15 to 20 mm length (for example, the pieces may have the following size distribution: 5-10% at >2360 μm; 1000 μm<80-90%<2360 μm; 10-15% at <1000 μm) prior to the aqueous extraction. This allows for an optimization of the extraction process, while at the same time avoiding the extraction of carrageenans in the first extraction step. In the method of the invention, preferably all carrageenans contained in the algae are subjected to the acidic hydrolysis.

The aqueous extraction is typically performed using water, in particular deionized water. Alternatively, it is also possible to use regular tap water, for instance, or water containing additives, such as e.g. glycerol, propane diol, ethanol or other polar solvents, or a buffer.

In an embodiment, the aqueous extraction comprises macerating the Mastocarpus stellatus in water, in particular in deionized water.

The aqueous extraction, in particular the maceration in water, may be performed at a suitable concentration of the Mastocarpus stellatus in the water.

In an embodiment, the aqueous extraction comprises macerating the Mastocarpus stellatus in water, in particular in deionized water, at a concentration of about 2 to 10%, more preferably at about 4 to 5%, for example at about 4.8%.

Throughout this disclosure, indications of percentages (%) refer to weight percentages (w/w) if not indicated otherwise.

The aqueous extraction, in particular the maceration in water, may be performed at room temperature. Alternatively, it is also possible to use a slightly higher or lower temperatures.

In an embodiment, the aqueous extraction comprises macerating the Mastocarpus stellatus at a temperature of about 10 to 30° C., more preferably at about 20° C.

The aqueous extraction, in particular the maceration in water, may be performed for a suitable period of time, depending on the temperature and/or concentration used.

In an embodiment, the aqueous extraction comprises macerating the Mastocarpus stellatus for about 5 to 60 minutes, more preferably for about 15 minutes.

In a particular embodiment, the aqueous extraction comprises macerating the Mastocarpus stellatus in water, in particular in deionized water, at a concentration of about 2 to 10%, more preferably at about 4 to 5%, at a temperature of about 10 to 30° C., more preferably at about 20° C., for about 5 to 60 minutes, more preferably for about 15 minutes.

The first liquid phase and the first solid phase may be separated using any suitable method. The preferred method may vary depending on the scale of the aqueous extraction, for example, but also on other factors, such as particle size, viscosity of the liquid phase, or the available equipment. Centrifugation, sieving, and/or filtration are particularly suitable methods.

In an embodiment, the first liquid phase and the first solid phase are separated by one or more of centrifugation, sieving, and filtration.

After the first phase separation, the first liquid phase may be stored under suitable conditions. For example, the first liquid phase may be stored at ambient temperature, protected from light, for a few hours (e.g. about 3-4 h) during the remaining extraction process. In addition, the first liquid phase may be stabilized by the addition of a suitable stabilizer, e.g. a (polar) solvent. For example, the first liquid phase may be stabilized by the addition of 20% of propane diol.

The acidic hydrolysis of the first solid phase is used, in particular, to hydrolyze carrageenans contained in the algae material.

The acidic hydrolysis of the first solid phase may be conducted at a suitable pH. Depending on the desired degree of hydrolysis, a slightly higher or lower pH may be selected.

In an embodiment, the acidic hydrolysis is conducted at a pH of about 1 to 4, more preferably at a pH of about 1.5 to 2.5, and most preferably at a pH of about 2.

The desired pH may be obtained by the addition of a suitable acid. Sulfuric acid, hydrochloric acid, citric acid and mixtures thereof are particularly well suited.

In an embodiment, the acidic hydrolysis comprises the addition of sulfuric acid, hydrochloric acid, citric acid or any mixture thereof.

The ratio of the Mastocarpus stellatus to the acid may also be adjusted depending on the desired degree of hydrolysis. A suitable ratio is, for example, about 3-6% of sulfuric acid, preferably about 4.5% of sulfuric acid.

The acidic hydrolysis is conducted at elevated temperature. Depending on the desired degree of hydrolysis, a higher or lower temperature may be selected.

In an embodiment, the acidic hydrolysis is conducted at a temperature of about 50 to 90° C., more preferably at a temperature of about 70 to 90° C., and most preferably at a temperature of about 80° C.

The duration of the acidic hydrolysis may also be adjusted depending on the desired degree of hydrolysis. The longer the duration, the lower the degree of polymerization.

In an embodiment, the acidic hydrolysis is conducted over a period of about 1 hour to 3 hours, more preferably of about 2 hours.

The acidic hydrolysis may be stopped by increasing the pH or lowering the temperature, for example.

In an embodiment, the method of the invention further comprises the step of adjusting the pH to a pH of about 4 to 8, more preferably to a pH of about 4.5 to 6, and in particular a pH of about 5, after the acidic hydrolysis.

The pH may be adjusted by the addition of a suitable base, for example a mineral base, such as sodium hydroxide.

After the acidic hydrolysis, and optionally adjusting the pH, a phase separation is performed in order to obtain a second liquid phase and a second solid phase. This phase separation may be achieved by any suitable means, for example by centrifugation, filtration, vibrating sieving, and/or wringing.

Therefore, in an embodiment, the phase separation in step (iii) comprises one or more of centrifugation, filtration, vibrating sieving, and wringing.

Optionally, a suitable stabilizer may be added to the second liquid phase prior to combining it with the first liquid phase. For example, the second liquid phase may be stabilized by the addition of a (polar) solvent, e.g. of 20% of propane diol. Preferably, the same stabilizer is used for both the first and the second liquid phase.

Finally, the first liquid phase and the second liquid phase are combined to obtain the Mastocarpus stellatus extract of the invention.

Optionally, the Mastocarpus stellatus extract of the invention may be filtered after combining the first and second liquid phase, for example up to 0.3 μm.

Optionally, the Mastocarpus stellatus extract of the invention may be heated after combining the first and second liquid phase, for example to about 60° C. This may reduce the pink color of the extract, presumably by destroying pigments, such as phycoerytrin.

The following table provides a comparison of the first liquid phase, the second liquid phase and the Mastocarpus stellatus extract of the invention:

| | First | Second | Mastocarpus stellatus |
|---|---|---|---|
| | liquid phase | liquid phase | extract of the invention |
| Final pH | 6.06-6.77 | 4.84-5.21 | 5.45-6.10 |
| Dry matter (%) | 0.98-1.51 | 2.19-2.43 | 1.63-1.78 |
| Floridoside (g/l) | 0.87-1.72 | 0.25-0.74 | 0.83-0.86 |
| Shinorine (g/l) | 0.13-0.20 | 0.03-0.07 | 0.07-0.11 |
| Total neutral sugars | No data | No data | 10.42 |
| (g/l) | | | |

The Mastocarpus stellatus extract may be used as such, or it may be diluted or concentrated, depending on the intended use and desired concentration.

The Mastocarpus stellatus extract of the invention may be stored at suitable conditions, for example at ambient temperature and preferably protected from light.

The present invention also relates to a Mastocarpus stellatus extract obtained by or obtainable by the method of the invention.

As will be appreciated by the person skilled in the art, as used herein, the term “obtainable from” means that the extract may be obtained from a plant or may be isolated from the plant, or may be obtained from an alternative source, for example by chemical synthesis or enzymatic production. Whereas the term “obtained” as used herein, means that the extract is directly derived from the plant source.

The Mastocarpus stellatus extract of the present invention has been found to have several advantageous cosmetic effects, which have been proven both by in vitro and clinical studies (see examples below).

In particular, it was found that the Mastocarpus stellatus extract is able to reduce a double chin, to enhance lipolysis, to increase skin elasticity, to stimulate collagen synthesis, to hydrate skin, to reduce the effects of skin aging and to prevent the effects of skin aging.

The Mastocarpus stellatus extract is advantageously topically applied to the skin.

In a further aspect, the present invention provides a cosmetic composition comprising the Mastocarpus stellatus extract of the present invention and a cosmetically acceptable excipient. The cosmetic composition of the present invention is intended for topical application.

The concentration of the Mastocarpus stellatus extract in the cosmetic composition should be chosen such that the desired effect is achieved.

Any excipients commonly used in the preparation of cosmetic preparations for use on the human skin may be employed in the present invention. Suitable excipients include, but are not limited to ingredients that can influence organoleptic properties, penetration of the skin, and the bioavailability of the Mastocarpus stellatus extract. More specifically, they include liquids, such as water, oils or surfactants, including those of petroleum, animal, plant or synthetic origin, such as and not restricted to, peanut oil, soybean oil, mineral oil, sesame oil, castor oil, polysorbates, sorbitan esters, ether sulfates, sulfates, betaines, glycosides, maltosides, fatty alcohols, nonoxynols, poloxamers, polyoxyethylenes, polyethylene glycols, dextrose, glycerol, digitonin, and the like.

The formulation for topical application to the skin may take any physical form. For instance, the cosmetic composition, and in particular the skin care composition, may be in the form of a liposome composition, mixed liposomes, oleosomes, niosomes, ethosomes, milliparticles, microparticles, nanoparticles and solid-lipid nanoparticles, vesicles, micelles, mixed micelles of surfactants, surfactant-phospholipid mixed micelles, millispheres, microspheres and nanospheres, lipospheres, millicapsules, microcapsules and nanocapsules, as well as microemulsions and nanoemulsions, which can be added to achieve a greater penetration of the Mastocarpus stellatus extract.

The cosmetic composition, and in particular the skin care composition, may be produced in any solid, liquid, or semi-solid form useful for application to the skin topically or by transdermal application. Thus, these preparations of topical or transdermal application include, but are not restricted to, creams, multiple emulsions, such as and not restricted to, oil and/or silicone in water emulsions, water-in-oil and/or silicone emulsions, water/oil/water or water/silicone/water type emulsions, and oil/water/oil or silicone/water/silicone type emulsions, micro-emulsions, emulsions and/or solutions, liquid crystals, anhydrous compositions, aqueous dispersions, oils, milks, balsams, foams, aqueous or oily lotions, aqueous or oily gels, cream, hydro-alcoholic solutions, hydro-glycolic solutions, hydrogels, liniments, sera, soaps, face masks, serums, polysaccharide films, ointments, mousses, pomades, pastes, powders, bars, pencils and sprays or aerosols (sprays), including leave-on and rinse-off formulations.

Thus, the present invention also provides a skin care composition.

The cosmetic composition of the invention may optionally further comprise other cosmetic active agents, for example anti-ageing, moisturizing or hydrating agents.

In an embodiment, the cosmetic composition of the invention further comprises an anti-ageing agent comprising a mixture of mannose-6-phosphate and mannose, as described in WO 2020/201185. The disclosure of WO 2020/201185 with respect to the cosmetic active agent and its embodiments and uses is herewith incorporated by reference.

In a further aspect, the present invention also provides a method of reducing a double chin, comprising the step of topically applying a Mastocarpus stellatus extract, and in particular the Mastocarpus stellatus extract according to the invention or the cosmetic composition according to the invention, to the double chin.

In a further aspect, the present invention also relates to the use of the Mastocarpus stellatus extract of the invention or of the cosmetic composition of the invention for reducing a double chin.

In a further aspect, the present invention also relates to the use of the Mastocarpus stellatus extract of the invention or of the cosmetic composition of the invention for enhancing lipolysis.

In a further aspect, the present invention also relates to the use of the Mastocarpus stellatus extract of the invention or of the cosmetic composition of the invention for increasing skin elasticity.

In a further aspect, the present invention also relates to the use of the Mastocarpus stellatus extract of the invention or of the cosmetic composition of the invention for stimulating collagen synthesis.

In a further aspect, the present invention also relates to the use of the Mastocarpus stellatus extract of the invention or of the cosmetic composition of the invention for hydrating skin.

In a further aspect, the present invention also relates to the use of the Mastocarpus stellatus extract of the invention or of the cosmetic composition of the invention for reducing the effects of skin aging.

In a further aspect, the present invention also relates to the use of the Mastocarpus stellatus extract of the invention or of the cosmetic composition of the invention for preventing the effects of skin aging.

The present invention is further illustrated by means of the following non-limiting examples:

### Example 1: Mastocarpus stellatus Extract

A suspension of 4.8% of dry matter of Mastocarpus stellatus in water was prepared (20 g of dried algae+396.67 g of osmosed water). The algae material was first cut into small pieces of 9 mm length with scissors and then macerated under stirring for 15 minutes at room temperature (22° C.). The biomass (=first solid phase) and supernatant (=first liquid phase) were separated by filtration (Whatman filter, 40 μm). 1,3-propanediol was added to the supernatant at a concentration of 20% of the total amount to obtain extract 1. This was then stored at room temperature and protected from light for about 3-4 hours, while extract 2 was prepared.

The insoluble part of the previous extraction (=first solid phase) was suspended again to 4.8% of dry matter in osmosed water. The suspension was then stirred, acidified to pH=2 with 96% sulphuric acid solution and then heated to 80° C. The acid hydrolysis lasted 2 h at 80° C. After 2 h, the suspension was cooled and neutralized to pH=5 with 10 M sodium hydroxide solution. The extract was centrifuged (4500 rpm, 20° C., 20 min), the supernatant was separated from the solid, and 1,3-propanediol was added to the supernatant (=second liquid phase) at a concentration of 20% of the total amount to obtain extract 2.

At the end, the two extracts 1 and 2 were combined, stirred and heated to reach 60° C. After 30 minutes of heating, the mixture was immediately filtered at 2.5 μm and 0.3 μm successively to obtain a Mastocarpus stellatus extract according to the invention.

The extract thus obtained had a pH of about 5.45, a dry mass content of about 1.63%, a Gardner color of about 2.1, and a floridoside content of about 0.86 g/l.

### Example 2: Transcriptomic Analysis on Human Fibroblasts

Normal human dermal fibroblasts (NHDFs) were seeded at 300,000 cells per well in a 6-wells plate. After 48 hours of culture in Dulbecco's Modified Eagle Medium supplemented with 10% FCS, NHDFs were rinsed two times with phosphate buffered saline (PBS) and allowed to rest in FCS-free medium overnight before stimulation. Cells were stimulated with Mastocarpus stellatus extract at 0.5% and compared to the untreated condition. After 6 hours and 18 hours of stimulation, total RNA was extracted by the Extract-all method (Rio D C, Ares M Jr, Hannon G J, Nilsen T W. Purification of RNA using TRIzol (TRI reagent). Cold Spring Harb Protoc. 2010 June;2010(6):pdb.prot5439. doi: 10.1101/pdb.prot5439. PMID: 20516177.). RNA quality was controlled and a reverse transcription was performed to obtain cDNA. RT-qPCR was made on specific plates designed to study transcriptomic expression of different genes involved in skin elasticity for NHDFs with 10 ng of cDNA per well. The results of gene expression obtained with fibroblasts were normalized according to PES1 (Pescadillo Ribosomal Biogenesis Factor 1), GADD45A (Growth Arrest and DNA Damage Inducible Alpha) and HMBS (hydroxymethylbilane synthase) housekeeping genes. The data are expressed in fold change relative to untreated condition.

### Results

In order to explore if Mastocarpus stellatus extract is able to improve skin elasticity, a transcriptomic study was conducted on fibroblasts treated with Mastocarpus stellatus extract at 0.5%, and the expression of various genes involved in skin elasticity was analyzed.

After 6 hours of treatment, the Mastocarpus stellatus extract significantly increased the expression of genes involved in elastic fibers organization, such as FBLN5, LOXL1 and MFAP2, by +34%, +32% and +10%, respectively. It also decreased the expression of HPSE, a gene coding for a protein involved in elastic fibers degradation, by −53%.

Interestingly, after 18 h of treatment with the Mastocarpus stellatus extract, the expression of FBLN5 and MFAP2 was still significantly increased by +24% and +24%, respectively, and the expression of HPSE was decreased by −40%.

The Mastocarpus stellatus extract also stimulated the expression of genes involved in extracellular matrix structure, such as COL3A1 and CD44 by +33% and +58%, respectively.

The results are summarized in the following table:

| | Improvement of skin elasticity - transcriptomic study | | | | | |
|---|---|---|---|---|---|---|
| | (fold change vs. untreated condition) | | | | | |
| Time | Pathway | Genes | Fold change | SEM | P value | |
| 6 h | Elastic fibres | FBLN5 | 1.344 | 0.01 | 0.0001 | *** |
| | organization | LOXL1 | 1.318 | 0.03 | 0.0014 | ** |
| | | MFAP2 | 1.103 | 0.03 | 0.0412 | * |
| | Degradation | HPSE | −1.534 | 0.06 | 0.0183 | * |
| 18 h | Elastic fibres | FBLN5 | 1.243 | 0.08 | 0.088 | # |
| | organization | MFAP2 | 1.244 | 0.03 | 0.008 | ** |
| | | MFAP5 | 1.063 | 0.02 | 0.081 | # |
| | Extracellular | COL3A1 | 1.327 | 0.1 | 0.073 | # |
| | matrix | CD44 | 1.579 | 0.21 | 0.098 | # |
| | Degradation | HPSE | −1.401 | 0.06 | 0.0267 | * |

This transcriptomic study confirms the Mastocarpus stellatus extract as a promising candidate for improving skin elasticity.

### Example 3: Proteome Analysis on Human Skin Explants

The impact of Mastocarpus stellatus extract on skin elasticity was further evaluated at the proteome level.

### Sample Preparation

Skin explants from a young donor (28 years) and from a mature donor (59 years) were maintained in survival in an air-liquid interface. The skin explants from the young donor were kept untreated while the skin explants from mature donor were topically treated with Mastocarpus stellatus extract at 1% and compared to the untreated condition. Treatments and medium (MIL217C from Biopredic International) were renewed every day for 5 days. After these 5 days of stimulation, the skin explants rinsed two times with PBS and were frozen at −80° C.

### Sample Treatment

Sheared tissue was added to a zirconia oxide bead mix and 700 μl of iST LYSE buffer (Deoxycholate, TCEP and Chloroacetamide) were added. Samples were lysed by two rounds of bead beating cycle. Homogenates were transferred into Diagenode protein extraction tube and co-extracted nucleic acid and organelles were sheared by micro cavitation (Bioruptor Pico, Diagenode). Proteins were solubilized, reduced and alkylated by boiling in iST LYSE buffer (Deoxycholate, TCEP and Chloroacetamide). Protein concentration was determined by the BCA method (Walker J M. The bicinchoninic acid (BCA) assay for protein quantitation. Methods Mol Biol. 1994;32:5-8. doi: 10.1385/0-89603-268-X:5. PMID: 7951748.). Peptide extracts were prepared according to iST method (in Stage Tips). 50 μg of proteins were digested by a mix of LysC and trypsin. Peptides were purified by mixed mode reverse phase cation exchanger SPE (Solid Phase Extraction; PreOmics GmbH), dried and solubilized in 100 μl of 3% acetonitrile−0.1% formic acid aqueous solution. Peptide concentration was determined using the BCA method.

### LC-MS/MS

300 ng of peptides were injected in triplicate for each sample. Chromatography was performed using Ultimate 3000 (Di-onex) equipment using a C18 (75 μm×50 cm, 2 μm material) column applying a gradient of 2.5% to 35% acetonitrile over 120 minutes at a flow rate of 300 nl/min after a 3 minutes trapping step on the precolumn. Data were acquired using a Q-Exactive (Thermo) mass spectrometer. MS scan was performed with a resolution of 70,000 and an accumulation time of 60 ms. MS/MS scan was performed with a resolution of 17,500 on the 10 most intense ions of each cycle with an accumulation time of 60 ms. 6545 cycles were performed, thus an average of 17 cycles per chromatographic peak.

### Protein Identification

Proteins were identified using the SEQUEST-HT algorithm (Tabb DL. The SEQUEST family tree. J Am Soc Mass Spectrom. 2015;26(11):1814-1819. doi:10.1007/s13361-015-1201-3) against a database gathering Human reference proteome and enzymes used for digestion mined from UNIPROT. Search parameters were: enzyme=trypsin (full); allowed miscleavage=2; precursor error tolerance=10 ppm; fragment error tolerance=0.02 Da; dynamic modification=oxidation (M), deamidation (N/Q); protein terminus modification=acetylation; static modification=carbamidomethyl (C).

False Discovery Rate (FDR) determination was made using the Percolator algorithm (Spivak M, Weston J, Bottou L, Kall L, Noble W S. Improvements to the percolator algorithm for Peptide identification from shotgun proteomics data sets. J Proteome Res. 2009 July;8(7):3737-45. doi: 10.1021/pr801109k. PMID: 19385687; PMCID: PMC2710313.).

All spectra reported with a confidence less than high by SEQUEST-HT, thus considered as not identified, were reprocessed by MS Amanda 2.0 algorithm against the same database as above. Search parameters were: enzyme=trypsin (full); allowed miscleavage=2; precursor error tolerance=10 ppm; fragment error tolerance=0.02 Da; dynamic modification=oxidation (M)(P), deamidation (N/Q); protein terminus modification=acetylation, Met-Loss, Met-Loss+Acetyl; static modification=carbamidomethyl (C). False Discovery Rate (FDR) determination was made using the Percolator algorithm.

### Protein Quantification

Data were processed using Minora and feature mapper for Proteome Discoverer 2.3 software (Thermo Fisher Scientific). Peak integration parameters were: Post acquisition recalibration=True (fine parameters); minimum trace length=5; minimum number of isotope=2; max Delta RT for isotope=0.2 min; PSM (Peptide-Spectrum match) confidence level for integration=High.

Chromatographic alignment parameters were: RT alignment=TRUE; parameter tuning=fine; maximum RT shift=5 min; mass tolerance 10 ppm. Feature mapping parameters were: RT tolerance=automatic; mass tolerance=automatic; S/N threshold=2.

Statistical analyses were performed using Precursors Ions quantifier node for Proteome Discoverer 2.4 software (Thermo Fisher Scientific).

General Quantification Settings were: Peptide to use=Unique+RAZOR (Unique=peptides that are not shared by different proteins or protein groups; RAZOR=peptides shared by multiple protein's groups but only used to quantify protein with the largest number of unique peptides and with the longest amino acid sequence); Consider Proteins Groups for Peptide Uniqueness=True; Reject Quan Results with Missing Channels=False. Precursor Quantification Settings were: Precursor Abundance Based on Area; Min number Replicate feature=50% (peptides must be detected in at least 50% of sample of one group to be used in quantification). Normalization settings: Total Peptide amount (Calculates the total sum of abundance values for each injection over all peptides identified, the injection with the highest total abundance is used as reference to correct abundance values in all other injections by a constant factor per injection, so that at the end the total abundance is the same for all injections.)

### Data Selection

A first selection was performed by selecting only the proteins significantly impacted by ageing (mature donor versus young donor) and reversed by Mastocarpus stellatus extract (treated mature donor versus untreated mature donor). In this selection of proteins, proteins involved in skin dermis structure and elasticity were identified: 5 proteins were highlighted.

### Results

First, the impact of skin-ageing on the proteome was evaluated by comparing a young donor (28 years old) with a mature donor (59 years old). A significant decrease in the expression of proteins involved in elastic fibers organization, such as MFAP4, TIMP1 and FBLN5 (−10%, −60% and −76%, respectively), and of other structural proteins of the dermis, such as COL1A1 and COL2A1 (−75% for each), was found. This confirmed the negative impact of skin-ageing on the dermis structure.

The results compared to the young donor are summarized in the following table:

Impact of ageing - proteomic study (fold change vs. young donor)

| Pathway | Genes | Fold change | P value | |
|---|---|---|---|---|
| Elastic fibres | MFAP4 | −1.098 | <0.0001 | *** |
| organization | FBLN5 | −1.756 | <0.0001 | *** |
| Dermis structure | TIMP1 | −1.596 | <0.0001 | *** |
| and protection | COL1A1 | −1.747 | <0.0001 | *** |
| | COL2A1 | −1.748 | <0.0001 | *** |

Secondly, the effect of the Mastocarpus stellatus extract on the mature donor, whose dermis structure is altered by ageing, was studied. It was found that the expression of the above five proteins, which were decreased compared to the young donor, was significantly increased after 5 days of treatment with the Mastocarpus stellatus extract. Indeed, regarding elastic fibers organization, the expression of MFAP4, TIMP1 and FBLN5 was increased by +64%, +23% and +95%, respectively. In the same way, the expression of COL1A1 and COL2A1 was increased by +17% and +14%, respectively, with the Mastocarpus stellatus extract.

The results compared to the mature donor are summarized in the following table:

Improvement of skin elasticity - proteomic
study (fold change vs. mature donor)

| Pathway | Genes | Fold change | P value | |
|---|---|---|---|---|
| Elastic fibres | MFAP4 | 1.641 | <0.0001 | *** |
| organization | FBLN5 | 1.953 | <0.0001 | *** |
| Dermis structure | TIMP1 | 1.229 | <0.0001 | *** |
| and protection | COL1A1 | 1.168 | <0.0001 | *** |
| | COL2A1 | 1.141 | <0.0001 | *** |

These results confirm at the protein level that the Mastocarpus stellatus extract is beneficial for dermis structure and elastic fibers organization.

### Example 4: Skin Biomechanical Properties Analysis

### Sample Preparation

Skin explants from a young donor (19 years) and from a mature donor (49 years) were maintained in survival in an air-liquid interface. The skin explants from young donor were kept untreated while the skin explants from mature donor were topically treated with Mastocarpus stellatus extract at 1% and compared to the untreated condition. Treatments and medium were renewed every day for 3 days. After these 3 days of stimulation, the skin explants were rinsed two times with PBS, cryopreserved in OCT™ compound Mounting medium and cryosectionned at 20 μm thickness.

### Young Modulus Analysis by Atomic Force Microscopy (AFM)

The Atomic Force Microscope used in this study is a Bioscope Resolve (Bruker) with an added epifluorescence microscope (Leica DMi8). This configuration allows the precise positioning of the AFM probe on the sample. This unique combination also allows acquiring correlative images from mechanical to fluorescent acquisitions.

QNM (Quantitative Nanomechanical Mapping) Peakforce® mode was used in this study. AFM probe harbors a 0.4 N/m theoretical spring constant and a curvature radius <10 nm. Before each use, probe deflexion sensitivity was measured on a sapphire and its spring constant was also calibrated by the thermal noise method (Kim Y, Mandriota N, Goodnight D, Sahin O. Calibration of T-shaped atomic force microscope cantilevers using the thermal noise method. Rev Sci Instrum. 2020 Aug. 1;91(8):083703. doi: 10.1063/5.0013091. PMID: 32872926; PMCID: PMC7413748.). Force measurements were performed in air (PBS 1X, Batch number: CP20-3404).

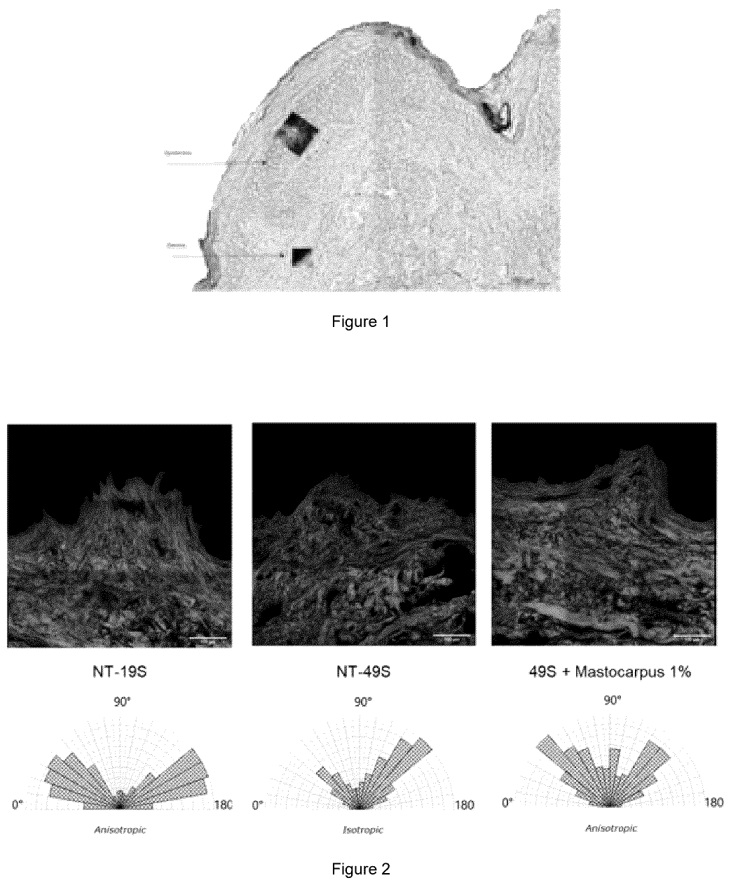

AFM measurement consists in the acquisition of Force-volume on the dermis as illustrated in FIG. 1. To each pixel of the image corresponds a Force-indentation curve, from which the elastic modulus (Ea) is extracted. There may be differences in the number of values from an area to another one, as some aberrant data are suppressed by the software.

### Collagen Fibre Organisation Analysis by Bi-Photonic Microscopy

A ZEISS LSM880 inverted confocal microscope was used for SHG (Second Harmonic Generation) imaging. The laser used was a Coherent biphoton pulsed Cameleon laser. The objective was a “C-Apochromat” water x40 objective. To image collagen, samples were excited at 900 nm wavelength and light was collected with a filter at 445 nm. 3 large images of 600 μm×600 uμm size were made per condition.

Data analysis was made using image J and analysis of mean grey value on 3 surfaces of 50×50 μm size per image.

Study of collagen fibre orientation was performed using the Rozeta software, on 3 areas of 50×50 μm per image.

### Results: Restoration of Skin Elasticity

Measurement of the elastic modulus evidenced the impact of skin-ageing on both the epidermis and the dermis. Indeed, there was a significant and strong increase of the elastic modulus in the 49-years old donor compared to the 19-years old donor. This means that the AFM probe needed a higher force to deform the mature skin compared to the young skin, translating a loss of skin suppleness with ageing.

The application of the Mastocarpus stellatus extract on mature skin surface, however, significantly reduced the elastic modulus in the epidermis and in the dermis, evidencing an improvement of skin suppleness. The results are summarized in the following table:

Skin suppleness (Elastic modulus in kPa)

| | Young | Mature | Mature skin + Mastocarpus |
|---|---|---|---|
| | skin | skin | stellatus extract (1%) |
| Average | 2.529 | 24.509 | 3.037 |
| SEM | 0.887 | 4.234 | 0.913 |
| P value vs. young | | <0.0001*** | |
| skin | | | |
| P value vs. mature | 0.05* | | <0.0001*** |
| skin | | | |

### Results: Restoration of Dermis Scaffold

Skin suppleness/rigidity properties are mainly induced by the dermis scaffold organization. In order to understand how the elastic modulus is impacted by ageing, the organization of collagen fibers in the dermis was studied.

It was found that collagen fibers in the young donor presented a 2D organization, meaning that the fibers were oriented in at least two directions, constituting a scaffold. Fibers in the dermis from the mature donor, on the other hand, presented a more isotropic organization, meaning that the fibers were mainly oriented in one direction, showing a loss of dermis scaffold. This change in collagen fibers orientation with age, leading to a loss of scaffold, can be directly linked to the loss of skin suppleness.

Interestingly, the topical application of the Mastocarpus stellatus extract on the mature donor skin explants led to a reorientation of the collagen fibers in a 2D organization. This 2D organization favors the reorganization of dermis scaffold, which could explain the improvement of the skin biomechanical properties shown by AFM. FIG. 2 shows the distribution of collagen fibers' orientation in the two dimensions analyzed in the pictures. For each direction, the longer the bars are, the more fibers are oriented in the respective direction.

### Example 5: Lipolytic Activity

Another factor involved in double chin formation is the accumulation of fatty tissue in the chin. In order to determine if the Mastocarpus stellatus extract was able to reduce the double chin volume by decreasing the fatty tissue content, mature adipocytes were embedded in a 3D matrix.

### 3D Culture of Human Mature Adipocytes

Mature adipocytes were obtained from subcutaneous adipose tissue coming from a woman aged 30 who had a Body Mass Index (BMI) of 28.7 kg/m² and gave her tissue under informed consent. Mature adipocytes were isolated from subcutaneous adipose tissue after digestion by collagenase. The isolated adipocytes were washed with a wash buffer and encapsulated in a peptidic hydrogel to form 3D adipocytes capsules of 25 μl in size. The formation of adipocytes capsules followed an internal standardized protocol to have about the same number of adipocytes between the capsules. Cells were then incubated for 24 h at 37° C. for stabilization. Treatments with Mastocarpus stellatus extract at 0.5% were initiated at D0 with a medium change; and the culture media were changed every day until 4 days of culture. At each culture media change, the cells secretions were collected, centrifuged and frozen at-80° C. for further analysis. Each culture condition was done in triplicate.

### Quantification of Extracellular Secretions of Glycerol and Adiponectin

The culture media after 72 h of treatment were collected. The concentrations of adiponectin and glycerol were evaluated by ELISA and colorimetric assays, respectively, using specific kits according to the manufacture's recommendations (adiponectin kit, Duoset, DY1065, R&D Systems; Glycerol kit, Randox, GY105).

### Results: Glycerol Release

Evaluation of glycerol released by adipocytes was performed in the adipocytes culture media after 72 h of treatment. Isoproterenol at 1 μM (positive control) significantly increased glycerol release by +196% through its lipolytic effect on adipocytes, validating this model. The Mastocarpus stellatus extract at 0.5% also had a significant effect, with +36% of glycerol release after 72 h of treatment, evidencing a lipolytic activity.

The results are summarized in the following table:

Glycerol release (% of untreated)

| | | | Mastocarpus stellatus |
|---|---|---|---|
| | Untreated | Isoproterenol | extract (0.5%) |
| Average | 100% | 295.6 | 135.7 |
| SEM | 9.673 | 17.51 | 4.983 |
| P value vs. untreated | | 0.05 * | 0.05 * |

### Results: Adiponectin Secretion

Adiponectin is another factor secreted by mature adipocytes. It was found that the Mastocarpus stellatus extract at 0.5% significantly increased the adiponectin secretion by +48% after 72 h of treatment, confirming its lytic activity on fatty tissue.

The results are summarized in the following table:

Adiponectin secretion (% of untreated)

| | Untreated | Mastocarpus stellatus extract (0.5%) |
|---|---|---|
| Average | 100% | 147.5 |
| SEM | 5.533 | 9.248 |
| P value vs. untreated | | 0.05 * |

### Example 6: Clinical Studies—Skin Biomechanical Properties, Double Chin Reduction and Improvement of V-shape

### Formulation

For the first set of clinical studies described below, a cosmetic formulation having the following INCI formula was used:

AQUA/WATER, CETHYL ALCOHOL, GLYCERYL STEARATE, PEG-75 STEARATE, CETETH-20, STEARETH-20, ISODECYL NEOPENTANOATE, MASTOCARPUS STELLATUS EXTRACT, GLYCERIN, PHENOXYETHANOL, DIMETHICONE, PHENOXYETHANOL, METHYL PARABEN, PROPYL PARABEN, ETHYL PARABEN, FRAGRANCE

In the placebo composition, the Mastocarpus stellatus extract was omitted.

In more detail:

| INCI | Active | Placebo |
|---|---|---|
| AQUA/WATER | 88.30 | 87.30 |
| CETHYL ALCOHOL, GLYCERYL STEARATE, | 5 | 5 |
| PEG-75 STEARATE, CETETH-20, STEARETH-20 | | |
| ISODECYL NEOPENTANOATE | 4.5 | 4.5 |
| MASTOCARPUS STELLATUS EXTRACT | 1 | — |
| GLYCERIN | 1 | 1 |
| PHENOXYETHANOL | 0.695 | 0.695 |
| DIMETHICONE | 0.3 | 0.3 |
| PHENOXYETHANOL, METHYL PARABEN, | 0.105 | 0.105 |
| PROPYL PARABEN, ETHYL PARABEN | | |
| FRAGRANCE | 0.1 | 0.1 |

### Panel Description

A single-center study with 44 volunteers with dry skin (corneometry measurement <70 a.u) and presenting wrinkles in the crow's feet and chin ptosis was carried out. Volunteers were divided into two groups of 22 volunteers each as follows:
Group 1: 22 women with an average age of 49±6 years, testing the cream containing Mastocarpus stellatus extract
Group 2: 22 women with an average age of 49±5 years, testing the placebo cream

The study was conducted according to the standard operating procedure of Bio EC and in compliance with the regulations established in “Guia para investigaciones con seres humanos” (Guidelines for Research on Human Beings) and the guidelines of the Scientific Committee on Consumer Safety (SCCS).

Volunteers applied cream containing 1% of Mastocarpus stellatus extract and the placebo cream, respectively, every day in the morning and in the evening for 28 days.

During this study, the biomechanical properties were analyzed using Cutometery® analysis and by fringes protection with AEVA HE® as described below.

### Skin Biomechanical Properties Measurement by Cutometer®

The measurement of the mechanical properties of the skin enables to assess the functional state of the elastic tissue structures (elastic fibers, curvature of the connective bundles, wrinkles of the stratum corneum) and the viscous-behaving tissue structures (interstitial fluids, internal adherences).

The study was performed using a Cutometer® MPA 580 by Courage & Khazaka. The measuring principle is based on the suction method: Negative pressure is created in the device and the skin is drawn into the cylindrical aperture (2 mm in diameter) of the probe. Inside the probe, the penetration depth is determined by an optical measuring system. Each suction phase is followed by a relaxing phase.

The following program was used in the present study:
Length of the cycle: 4 seconds (suction: 2 seconds; relaxation: 2 seconds)
Negative pressure: 450 millibars
Diameter of the chamber: 2 mm
Area measured: crow's feet

The resistance of the skin to be sucked up by the negative pressure and its ability to return to its original position were displayed as curves at the end of each measurement. From these curves, the parameters can be calculated:
During the suction phase, the deformation of the skin by the negative pressure first determines the elastic resistance and then the viscous component, which taken together are representative of the skin firmness.
During the relaxation phase, the immediate recovery of the skin determines sheer cutaneous elasticity, whereas the delayed return of the skin to its initial position measures the visco-elastic component.

The study focused on the following parameters:
R0 or Uf, which represents the amplitude of the skin during the suction phase: At equal pressure, the more flexible the skin, the greater the amplitude. Thus, this parameter evaluates the viscoelastic dispensability, or in other words, the firmness of the skin.
R5 represents the net elasticity (Ur/Ue): Elastic portion of the relaxation region (Ur) devided by the elastic portion of the suction region (Ue).

These parameters were measured at D0 and D28.

### Analysis by AEVA HE®: Focus Double Chin Volume (V-Shape Reshaping)

Based on a patented (U.S. Pat. No. 7,821,649) fringe projection unit combined with stereo imaging techniques, the AEVA-HE® system allows for different measurements, from wrinkles reduction to body reshaping. It is designed to quantify the efficacy of cosmetic, aesthetical and dermatologic products and treatments.

In particular, it can be used for assessing face sagging and double chin.

### Statistical Analysis

For the in vivo studies, a Shapiro Wilk test was used to verify whether the raw data followed the Gaussian Law. In case of normally-distributed data, the mean values were compared using either an unpaired or paired student t test. In case of non-normally-distributed data, a Wilcoxon (paired) and a Kruskal-Wallis test followed by a Mann-Whitney U (unpaired) test were used for paired data and unpaired data, respectively.

Whatever the statistical test used, results were considered significant as follows: #p<0.1, *p<0.05, **p<0.01 and ***p<0.001.

Concerning the analysis of the self-assessment questionnaire and daily log results, a Chi-square test was done (dichotomous analysis, which consists in comparing the number of associated answers).

### Results: Increased Skin Firmness and Elasticity

In a first clinical study, the skin biomechanical properties of the skin were measured after an application of a cream containing Mastocarpus stellatus extract™ at 1% or of a placebo cream on the face. The skin properties were evaluated using a Cutometer® after 28 days of application, measuring the R0 and R5 parameters.

### Effect on Skin Firmness

After 28 days of application, a decrease of the R0 parameter was observed, which indicates an increase of firmness with the Mastocarpus stellatus extract (−7%) in comparison to the placebo.

In addition, it was found that the effect of the Mastocarpus stellatus extract was significantly different from the placebo effect, showing a significant increase of firmness by up to 7 fold during the study, as can be seen from the following data:

| | | Cream with 1% Mastocarpus | Placebo | Active versus |
|---|---|---|---|---|
| | Firmness effect (R0) | stellatus extract (Active Cream) | Cream | Placebo |
| D 0 | Mean +/− SD | 0.290 ± 0.040 | 0.278 ± 0.035 | |
| | (arbitrary unit) | | | |
| D 28 | Mean +/− SD | 0.271 ± 0.029 | 0.275 ± 0.041 | |
| | (arbitrary unit) | | | |
| | Average variation vs | −7% | −1% | |
| | D 0 | | | |
| | Paired t test versus D 0 | ** p < 0.01 | ns | |
| | Unpaired t test | | | # p < 0.1 |

### Effect on Skin Elasticity

After 28 days of application, a significant increase of the R5 parameter was observed with the Mastocarpus stellatus extract in comparison to D0. With the placebo, a slight increase was showed relative to D0. Measurements further showed an improved skin elasticity (+8%) with the cream containing the Mastocarpus stellatus extract relative to the placebo cream as can be seen from the following data:

| | | Cream with 1% Mastocarpus | Placebo | Active versus |
|---|---|---|---|---|
| | Elasticity (R5) | stellatus extract (Active Cream) | Cream | Placebo |
| D 0 | Mean +/− SD | 0.348 ± 0.059 | 0.375 ± 0.044 | |
| | (arbitrary unit) | | | |
| D 28 | Mean +/− SD | 0.374 ± 0.042 | 0.386 ± 0.042 | |
| | (arbitrary unit) | | | |
| | Average variation vs D 0 | +8% | +3% | |
| | Paired t test versus D 0 | ** p < 0.01 | # p < 0.1 | |
| | Unpaired t test | | | # p < 0.1 |

### Results: Decrease of Double Chin Volume

AEVA HE® was used to measure the effect on double chin volume.

After 28 days of application of the active cream containing 1% of Mastocarpus stellatus extract, a clear reduction of the double chin volume was observed:

| | Double chin volume | Cream with 1% Mastocarpus | Placebo | Active versus |
|---|---|---|---|---|
| | [mm³] | stellatus extract (Active Cream) | Cream | Placebo |
| D 0 | Mean +/− SD | 587 ± 643.8 | 673.7 ± 278.8 | |
| | (mm³) | | | |
| D 28 | Mean +/− SD | 569 ± 647.3 | 683.0 ± 287.8 | |
| | (mm³) | | | |
| | Average volume | −18 mm³ | +9 mm³ | |
| | change vs D 0 | | | |
| | Paired t test versus D 0 | ns | # p < 0.1 | |
| | Unpaired t test | | | * p < 0.05 |

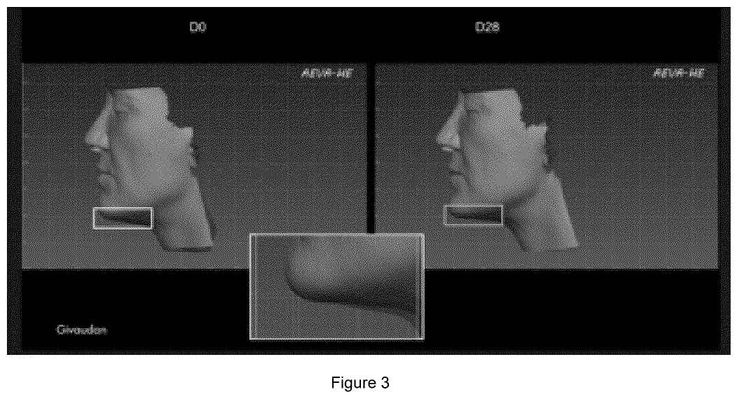

FIG. 3 shows illustrative photos of the AEVA HE® measurement of one of the volunteers applying the cream containing 1% of Mastocarpus stellatus extract.

### Results: Improvement of V-shape

During this study, the collagen level on the oval of the face was measured by SiAscope® after 56 days of a twice-daily application of a cream containing the Mastocarpus stellatus extract versus a placebo cream.

After 28 and 56 days of application, a significant increase of mean collagen of +2.4% and +4.2%, respectively, was observed with the Mastocarpus stellatus extract in comparison to D0. With the placebo, a slight increase was showed relative to D0 after 56 days. Comparing the product containing the Mastocarpus stellatus extract with the placebo, it was found that the Mastocarpus stellatus extract is able to improve collagen 3.4 times more after 28 days and 2.8 times more after 56 days than the placebo.

The data is shown in the following table:

| | | Cream with 1% Mastocarpus | Placebo | Active versus |
|---|---|---|---|---|
| | Collagen level | stellatus extract (Active Cream) | Cream | Placebo |
| D 0 | Mean +/− SD | 142.21 ± 9.1 | 142.63 ± 12.5 | |
| | (arbitrary unit) | | | |
| D 28 | Mean +/− SD | 145.67 ± 10.5 | 143.68 ± 11.6 | |
| | (arbitrary unit) | | | |
| | Average variation vs | +2.4% | +0.7% | |
| | D 0 | | | |
| | Paired t test versus D 0 | ** | ns | |
| | Unpaired t test | | | # p < 0.1 |
| D 56 | Mean +/− SD | 148.18 ± 11.5 | 144.72 ± 14.6 | |
| | (arbitrary unit) | | | |
| | Average variation vs | +4.2% | +1.5% | |
| | D 0 | | | |
| | Paired t test versus D 0 | *** | # | |
| | Unpaired t test | | | * p < 0.05 |

In addition, Line-Field Optical Coherence Tomography (LC OCT®) was used to illustrate the dermal fibrous network at the level of the facial oval at D0 and D56. LC-OCT® is a non-invasive skin imaging technique, combining the high resolution of confocal microscopy to take a picture of the face in order to visualize the effect of collagen.

After 56 days of application of the cream containing the Mastocarpus stellatus extract at 1%, the appearance of long collagen fibers on the V-shape of the face was clearly observed.

After 56 days of placebo cream application, on the other hand, no long collagen fibers were observed.

Furthermore, an elasticity test was carried out that consists in applying a mass of 20 g suspended by a thread and attached by strip to the oval of the face. This test was carried out at D0, D28 and D56.

It was found that the skin had a higher tonus and more firmness after 28 and 56 days of application of the cream containing the Mastocarpus stellatus extract at 1%.

For the placebo cream, no effect was observed after 28 and 56 days of application.

### Example 7: Clinical Studies-Skin Hydration

### Formulation

For the second set of clinical studies described below, a cosmetic lotion having the following INCI formula was used:

AQUA/WATER, MASTOCARPUS STELLATUS EXTRACT SODIUM BENZOATE

In the placebo lotion, the Mastocarpus stellatus extract was omitted.

In more detail:

| INCI | Active | Placebo |
|---|---|---|
| AQUA/WATER | 98.50 | 99.50 |
| MASTOCARPUS STELLATUS EXTRACT | 1 | / |
| SODIUM BENZOATE | 0.5 | 0.5 |

### Panel Description

A single-center study with 40 volunteers with dry skin on the cheek with a hydration level of <50 a.u was carried out. Volunteers were divided into two groups of 20 volunteers each as follows:
Group 1: 20 volunteers with an average age of 47±6 years, testing a lotion containing Mastocarpus stellatus extract
Group 2: 20 volunteers with an average age of 48±5 years, testing a placebo lotion

Volunteers applied a lotion containing 1% of Mastocarpus stellatus extract or a placebo lotion every other day for 56 days. The hydration was evaluated using Raman spectroscopy.

### Results

In order to evaluate the level of hydration on the forearm, the total water content in the Stratum corneum was measured in % by Raman spectroscopy after a twice-daily application for two months.

After 56 days of application of the lotion containing 1% of Mastocarpus stellatus extract, a significant increase of the total water content in the Stratum corneum of +41.9% was observed in comparison to D0. With the placebo, a significant increase was showed relative to D0 after 56 days.

In comparison, the lotion containing 1% of Mastocarpus stellatus extract showed an improvement of the skin hydration by +10.3% relative to the placebo after 56 days.

The data can be found in the following table:

| Mean total | 1% Mastocarpus stellatus extract | | | Placebo | | | |
|---|---|---|---|---|---|---|---|
| water | | Average | Paired | | Average | Paired | Unpaired |
| content in | Mean ± SD | variation | t test | Mean ± SD | variation | t test | t test vs. |
| SC TW (a.u.) | (arbitrary unit) | vs. D 0 | vs. D 0 | (arbitrary unit) | vs. D 0 | vs. D 0 | Placebo |
| D 0 | 29.47 ± 6.35 | | | 28.90 ± 9.52 | | | |
| T24 h | 32.51 ± 10.48 | +10.3% | ns | 30.06 ± 10.56 | +4.0% | ns | ns |
| D 28 | 37.21 ± 9.09 | +26.7% | ** | 34.73 ± 7.25 | +20.2% | * | ns |
| D 56 | 41.82 ± 7.12 | +41.9% | ** | 38.04 ± 8.81 | +31.6% | ** | * p < 0.05 |

### Example 8: Hyaluronidase Inhibition Test (In Tubo)

In presence of its natural substrate, the polymer of hyaluronic acid is lysed by hyaluronidase. The presence of enzyme inhibitors leads to a reduction of hydrolysis rate. The inhibitory effect of the tested extract on hyaluronidase activity was determined with turbidimetric method by measuring the amount of non-lysed substrate.

The assay was performed in a 96-wells microplate.

The following samples were tested:

| Sample: | Description: |
|---|---|
| Extract 1 | First liquid phase obtained in step (i) of the |
| | method of the invention |
| Extract 2 | Second liquid phase obtained in step (iii) of |
| | the method of the invention |
| Mastocarpus stellatus | Combination of first and second liquid phase as |
| extract of the invention | obtained in step (iv) of the method of the |
| | invention |

20 μl of each sample was mixed with 10 μl of Mcllvaine buffer (pH 4.6). 20 μl of hyaluronidase and 20 μl of hyaluronic acid were added.

A negative control composed of solvent, Mcllvaine buffer (pH 4.6) and hyaluronic acid was also tested, as well as a positive control that additionally included the hyaluronidase.

In addition, a blank sample containing the respective sample, the buffer and the enzyme was prepared and tested for each of the extract samples.

The samples were tested at various concentrations. Each diluted sample was run in triplicate.

A standard range of disodium cromoglycate (DSCG) was used as inhibitor reference.

The mixture was stirred and allowed to stand for 40 min at 37° C.

Finally, 180 μl of CTAB (cetrimonium bromide) was added to all wells to precipitate non-hydrolyzed hyaluronic acid polymer.

The mixture was incubated for 20 min at room temperature, and the optical density (OD) at 600 nm was measured using a spectrophotometer.

The percentage of inhibition is calculated as follows:

$$\% Inhibition = \frac{OD ( sample ) - OD ( blank sample )}{OD ( negative control ) - OD ( positive control )} * 100 \%$$

Based on these measurements, the IC₅₀ was calculated, i.e. the sample concentration at which the hyaluronidase activity was inhibited by 50%. The results are shown in the following table:

| Sample: | IC₅₀: |
|---|---|
| Extract 1 | 10.8 μl/ml |
| Extract 2 | 8.4 μl/ml |
| Mastocarpus stellatus extract of the invention | 4.5 μl/ml |

As can be seen, the IC50 for the Mastocarpus stellatus extract of the invention is significantly lower than those of Extracts 1 and 2, revealing a synergistic effect observed in the extract of the invention.

### Example 9: Anti-glycation Activity Test (In Tubo)

Glycation is a non-enzymatic chemical reaction that can take place in the heart of the dermis. The glucose molecules react with proteins, which leads to disorganization of the dermis (glycated proteins). Glycated proteins accumulate because they cannot be eliminated. Glucose is fixed around the collagen and elastin fibers, which will stiffen and eventually break (loss of elasticity of the skin). This process is irreversible.

The anti-glycation activity test was carried out in a 96-wells microplate.

The same samples as in Example 8 were tested, again at various concentrations. Each diluted sample was run in triplicate.

A negative control containing sodium phosphate buffer and BSA (bovine serum albumin) and a positive control containing sodium phosphate buffer, BSA and ribose were also tested.

Finally, two blank samples were prepared and tested for each of the extract samples:
with phosphate buffer, BSA and extract
with phosphate buffer, ribose and extract

A standard range of aminoguanidine was used as inhibitor reference.

40 μl of each sample was mixed with 50 μl of BSA and 10 μl of ribose.

The mixture was stirred and incubated for 17 h at 37° C. The fluorescence (Fl) was determined with λ_{excitation}=340 nm and λₑₘᵢₛₛᵢₒₙ=420 nm.

The anti-glycation activity was calculated as follows:

$$\% Inhibition of AGEs formation = [ 1 - \frac{Fl ( sample ) - Fl ( blank sample ( ribose or BSA ) )}{Fl ( positive control ) - Fl ( negative control )} ] * 100 \%$$

Based on these measurements, the IC₅₀ was calculated, i.e. the sample concentration at which the AGEs formation was inhibited by 50%. The results are shown in the following table:

| Sample: | IC₅₀: |
|---|---|
| Extract 1 | 180.4 μl/ml |
| Extract 2 | 177.4 μl/ml |
| Mastocarpus stellatus extract of the invention | 39.4 μl/ml |

The above results reveal a strong synergistic effect observed for the extract of the invention.

1. A method of preparing a Mastocarpus stellatus extract, comprising the steps of:
i) aqueous extraction of Mastocarpus stellatus to obtain a first liquid phase and a first solid phase;
ii) acidic hydrolysis of the first solid phase at elevated temperature;
iii) phase separation to obtain a second liquid phase and a second solid phase; and
iv) combining the first liquid phase and the second liquid phase to obtain the Mastocarpus stellatus extract.

2. The method according to claim 1, wherein the Mastocarpus stellatus is dried and/or cut and/or ground prior to the aqueous extraction.

3. The method according to claim 1, wherein the aqueous extraction comprises macerating the Mastocarpus stellatus in water, in particular in deionized water, at a concentration of about 2 to 10%, at a temperature of about 10 to 30° C. for about 5 to 60 minutes.

4. The method according to claim 1, wherein the first liquid phase and the first solid phase are separated by one or more of: centrifugation, sieving, and filtration.

5. The method according to claim 1, wherein the acidic hydrolysis is conducted at a pH of about 1 to 4.

6. The method according to claim 1, wherein the acidic hydrolysis is conducted at a temperature of about 50 to 90° C.

7. The method according to claim 1, further comprising the step of adjusting the pH to a pH of about 4 to 8, after the acidic hydrolysis.

8. The method according to claim 1, wherein the phase separation in step (iii) comprises one or more of: centrifugation, filtration, vibrating sieving, and wringing.

9. A Mastocarpus stellatus extract obtained by or obtainable by the method according to claim 1.

10. A cosmetic composition comprising the Mastocarpus stellatus extract according to claim 9 and a cosmetically acceptable excipient.

11. A cosmetic composition according to claim 10, which is a skin care composition.

12. A method of reducing a double chin, comprising the step of topically applying a Mastocarpus stellatus extract according to claim 9 to the double chin.

13. A method of reducing a double chin, for enhancing lipolysis, for increasing skin elasticity, for stimulating collagen synthesis, for hydrating skin, for reducing the effects of skin aging and/or for preventing the effects of skin aging, compromising the step of utilizing the Mastocarpus stellatus extract of claim 9 on skin.

14. The method according to claim 3, wherein the aqueous extraction comprises macerating the Mastocarpus stellatus in water, in particular in deionized water, at a concentration of about 4 to 5%.

15. The method according to claim 3, wherein the aqueous extraction comprises macerating the Mastocarpus stellatus in water, in particular in deionized water, at a temperature of about 20° C.

16. The method according to claim 5 wherein the acidic hydrolysis is conducted at a pH of about 1.5 to 2.5.

17. The method according to claim 6, wherein the acidic hydrolysis is conducted at a temperature of about 70 to 90° C.

18. The method according to claim 7, further comprising the step of adjusting the pH to a pH of about 4.5 to 6, after the acidic hydrolysis.

19. A method of reducing a double chin, for enhancing lipolysis, for increasing skin elasticity, for stimulating collagen synthesis, for hydrating skin, for reducing the effects of skin aging and/or for preventing the effects of skin aging, comprising the step of utilizing the cosmetic composition of claim 10 on skin.
